(19) United States
(12) Patent Application Publication
(10) Pub. No.: US 2025/0049965 A1
(43) Pub. Date: Feb. 13, 2025

(54) ULTRASOUND GEL

(71) Applicant: Claudio Bandini, Bergamo (IT)

(72) Inventor: Claudio Bandini, Bergamo (IT)

(21) Appl. No.: 18/718,460

(22) PCT Filed: Dec. 16, 2022
(86) PCT No.: PCT/IB2022/062360

§ 371 Date: Jun. 11, 2024

(30) Foreign Application Priority Data
Dec. 16, 2021 (IT) 102021000031556

(51) Int. Cl. A61K 49/22

(52) U.S. Cl. CPC A61K 49/226

(57) ABSTRACT

The invention concerns an ultrasound conducting gel comprising a disinfectant component, wherein the disinfectant is non-alcoholic and non-volatile. Uses of the ultrasound conducting gel as an ultrasound transmitting agent for ultrasound equipment, for laser equipment and for high intensity pulsed light equipment are also described.

### FIELD OF THE INVENTION

The invention concerns an ultrasound conducting gel comprising a non-alcoholic or volatile disinfectant component.

Uses of the conducting gel as an ultrasound transmitting agent for ultrasound equipment, for laser equipment and for high intensity pulsed light equipment are also described.

### BACKGROUND ART

Ultrasound scan, or echotomography is a substantially non-invasive medical diagnostic investigation method that uses ultrasounds and is based on the principle of transmission of the ultrasound waves and of the transduction of the signal generated by them.

To date, it represents in a narrow sense the most widely used diagnostic investigation and minor interventional technique in medical practice, not least because, in addition to being modestly priced, it offers often satisfactory diagnostic responses within a short timeframe and, moreover, does not expose the patient to ionising radiations.

For all the reasons set out above, it is therefore used by any doctor at his/her discretion, both in public and private facilities, such as hospitals and private clinics and/or those affiliated with the national healthcare system.

From a practical point of view, the ultrasound scan is performed in most cases by using a transducer (probe) which, after the necessary application of the ultrasound gel, is placed in contact with the patient's skin in order to have a computerized and resolution image of the organs subject to analysis or to interventional procedure.

The gel is crucial in this process because, by substantially eliminating the air between the probe and the patient, it allows the ultrasounds to pass through efficiently and makes it possible to perform the ultrasound scan. The artefacts created by the air hinder the transmission of the ultrasounds; for example, the contrast medium for contrast-enhanced ultrasound scan itself contains air bubbles that are seen as hyperechogenic and therefore shine during the examination. It should be specified that at present, the main function of the ultrasound gel is precisely to reduce as much as possible the interference created by the air as the ultrasounds pass through.

However, the ultrasound gels currently on the market (both single-dose in sachets and in jars or bottles) are composed of thickeners, water and some dyes and have the sole function, described above, of creating a suitable interface between the ultrasound probe and the patient to allow ultrasounds to pass through, but they do not include any additives that are bacterial disinfectants or denaturalising agents against viruses.

The above refers to all types of gel used in the diagnostic-interventional ultrasound process currently on the market.

The single-dose ultrasound gel, which is used in the wards where it is necessary to ensure the maximum possible disinfection (i.e. paediatric intensive care) is made sterile, precisely to try to minimize the translocation of viruses and bacteria, but without per se disinfecting elements that neutralize viruses or bacteria.

It follows from the above that all the gels currently used present a considerable risk of transmission of viruses and bacteria, both operator-to-patient and patient-to-patient (though the use of ultrasound or gel), mainly due to (i) the contact with air; (ii) to a lack of care in the practice while handling the gel by doctors, nurses and healthcare operators, that is third parties; and, finally (iii) to an inadequate disinfection of the equipment attributable to the subjects referred to in point under (ii).

That said, it is first of all important to emphasize how both gels in single-dose sachet and those in jars or bottles can become a vehicle for the transmission of viruses and bacteria once they are exposed to prolonged contact with the air.

From this point of view, it is undeniable that often the gel jar or sachet is left open by the healthcare operator with the consequence that the gel, if contaminated, can transmit viruses and bacteria both on the personal protective equipment worn by the operator (i.e. gloves) and on the ultrasound medical probe and, consequently, on the patient.

This circumstance is obviously more likely to occur when the gel is handled by non-healthcare personnel (who can treat it as an extension of the ultrasound equipment itself) and becomes even more serious in the event of lack of care when using the gel in the single-dose sachet gel which, as mentioned above, is used in vulnerable wards where the need for sterility is even greater.

Therefore, in such wards, the lack of a per se disinfectant device forces the doctor or the third party to disinfect all the medical instrumentation used, especially the probe, as accurately as possible.

Indeed, if this were not the case, there would be a real risk that a gel residue, even minimal, left for a deficit of disinfection on a probe previously used on a compromised patient, would transfer a pathogen to another patient and, in the case of a pathogen resistant to antibiotic therapy, the risk of transmission might be extended to the entire ward.

A danger which, as anticipated, would be minimized by using the gel of the present invention.

Notwithstanding the above, the single-dose ultrasound gel in sachet also presents these additional criticalities: (i) by definition it can be used only once, resulting in waste of materials and increased costs and (ii) the application thereof on the probe by healthcare personnel equipped with an individual medical protection device (gloves) is cumbersome.

The criticalities set out above linked with the use of the gel commonly on the market also apply in the case of invasive ultrasound procedures, such as needle aspiration, which by now also envisage using the ultrasound gel for the most part, given that the povidone iodine, used in a residual way, not only fails to create the appropriate interface to the procedure because it is too liquid, but can also cause severe adverse reactions, up to the anaphylactic shock in patients allergic to iodine. For reasons of clarity it should be pointed out that povidone iodine is still used in some practises, which however can expose the patient to a rather severe allergic reaction if they are intolerant to iodine. Besides that, it is not a device suitable for the transmission of ultrasounds, because it is too liquid and not easy to handle.

In conclusion, since, as extensively set out above, there is currently no ultrasound gel that performs also a disinfectant function in addition to the canonical ultrasound transmitting function and which, by effect thereof, prevents patients and healthcare operators from incurring into viral and/or bacterial infections, the aim of the present invention is to provide an innovative ultrasound gel that allows to overcome the criticalities set out above while retaining an excellent ultrasound transmission, in particular by minimizing: (I) the translocation and the transmission of viruses and bacteria between operators and patients (and vice versa), or between patients and patients, both in the case of poorly disinfected ultrasound equipment and in the case of contamination of the gel due to contact with the air; (II) the use of pasteurized gel sachets, which can be replaced in their entirety by the use of the present gel, saving on materials and minimizing the risk of contaminations in already vulnerable wards; (Ill) human error thus making the ultrasound investigation safe even when it were performed with apparatuses that have not been diligently disinfected or with individual protection equipment that is not intact.

To this it should be added that the present gel makes invasive procedures safer and more agile not only because it minimizes the translocation of viruses and/or bacteria in procedures characterized by a high risk of contamination, but also because it makes the use of povidone iodine definitively obsolete which, as anticipated, not only exposes the patient to even serious adverse reactions because of its liquid composition, but also transmits the ultrasounds badly.

### SUMMARY OF THE INVENTION

The present invention originates from having identified an ultrasound conducting gel comprising, preferably consisting of a gelling component, a disinfectant component, disodium EDTA and water, wherein said disinfectant component is a non-alcoholic and non-volatile disinfectant.

In particular, this gel allows to minimize the issue of contamination by viruses and bacteria and of the need to use single-dose sachets which, as seen, can also be carriers of translocation of infectious agents when they are left in contact with air.

Under a second aspect, the present invention describes the use of the ultrasound conducting gel as an ultrasound transmitting agent for ultrasound equipment.

In accordance with what has been described so far and under a third aspect, the present invention describes the use of the ultrasound conducting gel, as a transmitting agent for laser equipment.

Under a fourth aspect, the present invention describes the use of the ultrasound conducting gel, as a transmitting agent for high intensity pulsed light (IPL) equipment.

Under a fifth aspect, the use of the ultrasound conducting gel, to prevent the transmission of bacteria, viruses and fungi, preferably said virus is the SARS-CoV-2 virus is described.

The dependent claims describe further embodiments of the invention.

### DESCRIPTION OF THE DRAWINGS

The invention will now be described in detail and with reference to the following accompanying figures, wherein:

FIG. 1 shows the photographic images of an ultrasound scan (A) and a Doppler ultrasonography (B) which have been performed using a commercially available ultrasound gel, without a disinfectant component;

FIG. 2 shows the photographic images of an ultrasound scan (A) and a Doppler ultrasonography (B) which have been performed using the ultrasound gel described in the present invention, comprising the disinfectant component.

### DETAILED DESCRIPTION OF THE INVENTION

The invention concerns an ultrasound conducting gel comprising, preferably consisting of a gelling component, a disinfectant component, a humectant component, a base, disodium EDTA and water, wherein said disinfectant component is a non-alcoholic and non-volatile disinfectant, and wherein:
said gelling component is in an amount in the range of 0.5 to 4% w/w,
said disinfectant component is in an amount in the range of 0.01 to 10% w/w, and
said disodium EDTA is in an amount in the range of 0.01 to 0.3% w/w.

The conducting gel is essentially free of air and other gases, which in the form of bubbles do not allow an effective ultrasound signal transmission. In fact, air is not a good conductor for mechanical sound waves and opposes resistance that must be eliminated with a gel as here described.

In a second embodiment, the present invention concerns an ultrasound conducting gel consisting of a gelling component, a disinfectant component, disodium EDTA, a humectant, a base, a dye and water, wherein said disinfectant component is a non-alcoholic and non-volatile disinfectant.

In the present invention when the following definitions are used:
“ultrasound conducting gel” it is intended to mean a gel for medical applications in the diagnostic and interventional field that allows it to act as a coupling interface between the ultrasound transducer, laser and pulsed light (the probe) and the patient's skin. The gel is for external use and has the purpose of allowing an optimal transmission of the ultrasounds from the instrument and through the patient's skin;
“gelling component” it is intended to mean a material of polymeric origin for medical use that allows the transmission from the probe to the patient's skin, eliminating the air that is interposed, such as for example the polyacrylic acid polymer (PPA) known as Carbomer or Carbomer 940; and
“disinfectant component” it is intended to mean a material that allows to reduce the amount of microorganisms, such as bacteria, viruses, and fungi, by controlling the risk of infection. In the gel of the present invention it is essential that the disinfectant component is non-alcoholic and non-volatile. Examples are hydrogen peroxide, quaternary ammonium compounds, especially those containing long alkyl chains, such as for example benzalkonium chloride, benzethonium chloride, cetrimonium, cetrimide and tetraethylammonium bromide. It is essential that the disinfectant component is not volatile, as are the alcohol-based disinfectants, which evaporate and dry the gel making it unusable for the purpose.

The definition:
humectant component means a hygroscopic substance capable of absorbing or retaining humidity in order to transfer it to another substance with which it is in contact. Examples of humectant or wetting agents with hygroscopic activity are: glycerol, sorbitol or low molecular weight hyaluronic acid, and other substances capable of capturing and retaining water; and
base means for example sodium hydroxide or other bases with similar characteristics that the average expert in the art would use as an alternative.

The gel of the present invention makes it possible to avoid the issue of contamination by viruses and bacteria and of the need to use single-dose sachets, with consequent advantages for operators in terms of practice safety and cost savings.

Advantageously, the gel of the present invention improves the safety window for the patient and the safety window for the operator (just think of a broken glove). In addition, the components of the gel allow to guarantee diagnostic quality and adequate disinfecting power with components that do not denature with UV, at temperature >25 degrees and that are safe (avoiding perfuming agents or other polymers).

The stability of the components of the gel is extremely important, just think that almost all ultrasound machines have a housing chamber for the gel bottle that allows it to be heated, to increase the compliance of the patient (paediatric and adult) with the examination. The gel of the present invention, having the composition indicated above is not altered and this temperature does not alter its disinfecting power or the viscosity thereof allowing a safe and diagnostic examination.

The technical novelty also lies in obtaining a viscosity similar to disinfectant-free common gels, which is absolutely necessary for the correct transmission of the ultrasounds for diagnostic purposes.

The gel of the present invention could be contained in transparent bottles, kept at room temperature as the chosen disinfectant is not UV sensitive and does not lose its disinfecting power in the common temperature ranges.

Furthermore, the gel according to the invention, being a safe gel which allows not using the probe cover device, allows safe use on newborns, on patients to be subjected to ultrasound-guided invasive procedures and in patients with surgical wounds, avoiding adverse reactions.

In a preferred embodiment, the ultrasound conducting gel according to the present invention has a gelling component in an amount in the range of 0.5 to 4% w/w, a disinfectant component in an amount in the range of 0.01 to 10% w/w and disodium EDTA in an amount in the range of 0.01 to 0.3% w/w.

In a further preferred embodiment the ultrasound conducting gel according to the present invention has a gelling component in an amount in the range of 0.5 to 2% w/w, a disinfecting component in an amount in the range of 0.01 to 2% w/w and disodium EDTA in an amount in the range of 0.01 to 1% w/w.

In a still more preferred embodiment, the ultrasound conducting gel according to the present invention has as a gelling component a polymer of the polyacrylic acid, such as for example Carbomer 940, as a disinfectant component benzalkonium chloride, the humectant component is glycerol and the base is sodium hydroxide.

Advantageously, benzalkonium chloride is not UV-sensitive and does not lose its disinfecting power in the common temperature ranges. The persistence through the gel of the disinfecting agent benzalkonium chloride at this concentration on the skin and on the probe increases its disinfecting power with respect to an aqueous solution, it allows reducing the use of chemicals for the disinfection of the probes and equipment and the persistence of the gel at the specific concentrations on the probe after the examination would not affect the safety of the following examination. It is further noted that the ultrasound conducting gel described herein may further comprise one or more components such as for example a dye.

In a more preferred embodiment, the ultrasound conducting gel has the composition: 1.5% w/w Carbomer 940, 10% w/w glycerol, 0.1% w/w dye, 0.2% w/w benzalkonium chloride, 0.05% w/w disodium EDTA, and wherein said water is deionized water.

The preferred embodiment allows to obtain a composition that improves both the safety of the operator and the safety of the patient guaranteeing an excellent diagnostic quality (precisely because it has no other components that are unnecessary to the medical procedure and that hinder it). To date, gels with characteristics same as or similar to the gel of the present invention are not available on the market.

Attempts to obtain ultrasound gels with disinfectant components have so far been unsatisfactory as they are not very viscous and have poor ultrasound transmission, in addition to the fact that some of them are harmful, and therefore the scientific community cannot consider them suitable for ultrasound investigation. If this were not the case, the disinfectant-free gels for ultrasound scan used by the radiological and non-radiological medical community would no longer exist, given the major issue of hospital-borne infections both among operators and between operator and patient and between patient and patient.

Under a second aspect, the present invention describes the use of the ultrasound conducting gel as an ultrasound transmitting agent, in particular for ultrasound equipment.

And indeed, the gel object of the present invention is to be used for the performance of diagnostic examinations carried out with ultrasound instruments (ultrasound scan, echocardiography) and in physiotherapy during treatments with ultrasound equipment equipped with contact heads and in the use of laser and pulsed light machinery.

In accordance with what has been described so far and in a third aspect, the present invention describes the use of the ultrasound conducting gel as a transmitting agent for laser equipment.

Under a fourth aspect, the present invention describes the use of the ultrasound conducting gel, as a transmitting agent for high intensity pulsed light (IPL) equipment.

Gels suitable for ultrasound scan (ultrasound transmission) are also always suitable for IPL and laser techniques, which do not need transduction for diagnostic purpose like the ultrasounds. If the gel conducts the ultrasounds very well, it will do the same for IPL and laser.

Under a fifth aspect, the use of the ultrasound conducting gel, to prevent the transmission of bacteria, viruses and fungi is described. In particular, it has been surprisingly found that the gel described in the present invention blocks the transmission of the SARS-CoV-2 virus.

Currently, the onset of a new human coronavirus, SARS-CoV-2, has become a global health issue. Human-to-human transmissions have been described with incubation times between 2-10 days, facilitating its diffusion through droplets, hands or contaminated surfaces. Human coronaviruses such as the severe acute respiratory syndrome (SARS) coronavirus, the Middle East respiratory syndrome (MERS) coronavirus, or the endemic human coronaviruses (HCoV) can persist on inanimate surfaces such as metal, glass, or plastic for up to 9 days, but they can be efficiently inactivated by disinfection procedures. Another advantage that is absolutely not to be underestimated is that this ultrasound gel prevents the transmission of this new human coronavirus.

The following Embodiment examples of the present invention are given below by way of illustration.

### Examples

### Example 1: Preparation of the Gel

An example of gel of the invention is prepared as follows:
Step 1: Carbomer 940, deionized water, glycerol and the dye were introduced into a first container equipped with a magnetic or mechanical stirrer. The resulting suspension (#1) is allowed to stir for 30 minutes at room temperature, so as to evenly disperse the polymer in the solution. The amounts of the different ingredients are indicated in Table 1.
Step 2: benzalkonium chloride, disodium EDTA and the second portion of deionized water were introduced into a second container equipped with a magnetic stirrer in the amounts indicated in Table 1. The mixture was allowed to stir at room temperature for a few minutes, until complete dissolution of the components in water, obtaining a solution (#2).
Step 3: Solution #2 was added to the suspension #1. The mixture was allowed to stir at room temperature for another 15 minutes.
Step 4: The mixture obtained from Step 3 was neutralized to pH=7 by adding an 18% NaOH solution dropwise. Vigorous stirring is necessary for minimizing the amount of trapped air and obtaining a clear, viscous gel.

| | |
|---|---|
| Ingredients | % w/w |

Part #1

| | |
|---|---|
| CARBOMER 940 | 1.5 |
| (Carbopol, Carboxyvinyl Acid, Gelling agent) | |
| Glycerol | 10 |
| Dye (e.g. Acid Green 25) | 0.1 |
| Deionized water | 77 |

Part #2

| | |
|---|---|
| Benzalkonium chloride | 0.2 |
| Disodium EDTA | 0.05 |
| Deionized water | 8 |

Part #3

| | |
|---|---|
| Sodium hydroxide (18% aqueous solution) | q.s. up to pH = 7.0 |
| Deionized water | q.s. up to 100.0% |
| Total: | 100 |

### Example 2: Use of the Gel

The gel prepared according to Example 1 was compared from a technical point of view with a commonly used gel for ultrasound scan, to verify whether the presence of the disinfectant component (in this case benzalkonium chloride) caused ultrasound conductivity issues and consequently affected the diagnostic value of the ultrasound investigation.

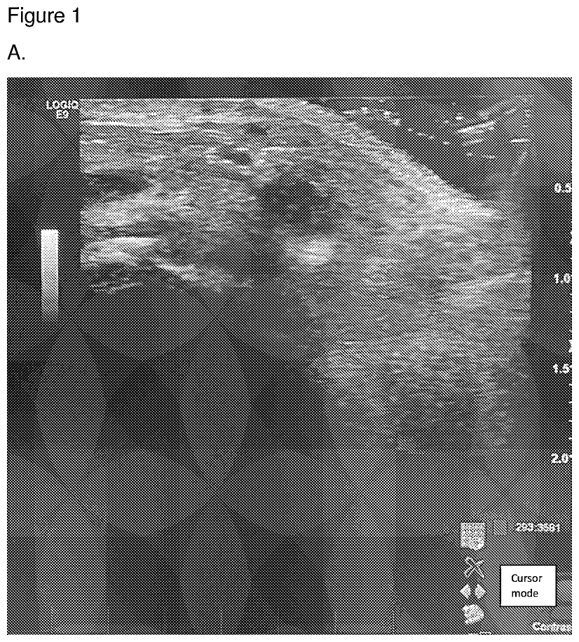

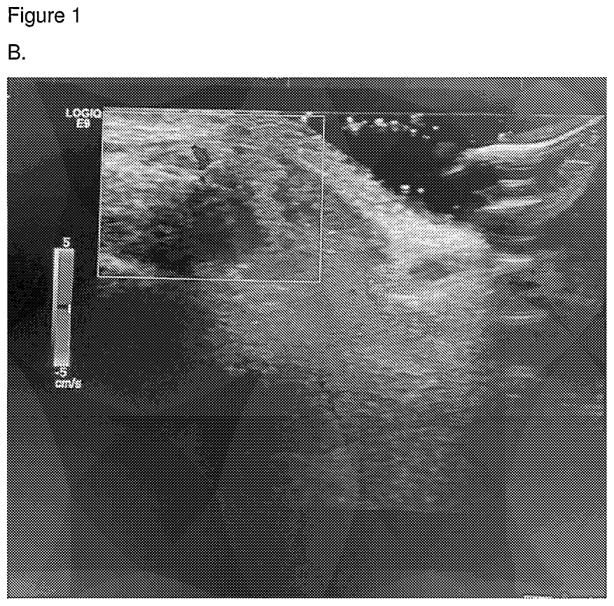

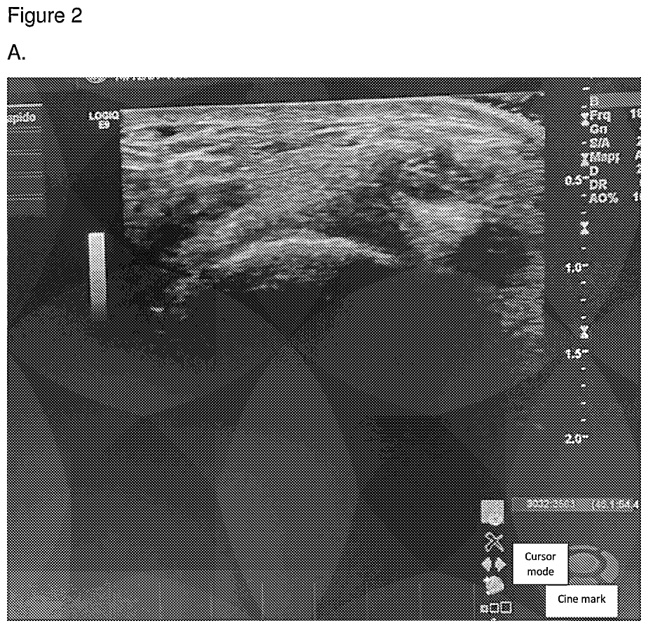

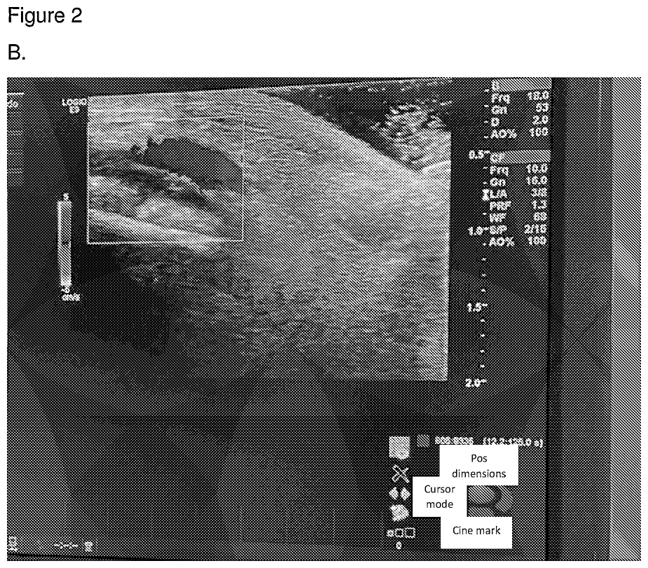

In detail, an ultrasound scan and a Doppler ultrasonography were performed on the same patient using first the gel currently on the market (see FIGS. 1A and 1B) and, subsequently, the gel according to the present invention (see FIGS. 2A and 2B). Specifically, FIG. 1 shows the images of the ultrasound scan (FIG. 1A) and of the Doppler ultrasonography (FIG. 1B) that have been performed with the commonly used and disinfectant-free gel.

For comparison, the images of an ultrasound scan (FIG. 2A) and of a Doppler ultrasonography (FIG. 2B) that have been performed with the gel prepared as described in Example 1 are shown in FIG. 2.

From a diagnostic and radiological point of view, the images of FIG. 1 and FIG. 2 are superimposable images.

The photographs were obtained with the free, prior and informed consent of the patient.

From the detailed description and from the above Examples, the advantages achieved by the gel of the present invention are evident. In particular, this gel has proved to be particularly suitable for medical applications that require an ultrasound transmission from the probe to the patient's skin.

This gel, having the same technical characteristics as common gels for ultrasound scans but, at the same time, the added advantage of the disinfectant function, can therefore be used both for all types of diagnostic examinations and for interventional procedures that involve ultrasounds, facilitating personnel in cleaning and maintaining the probe under conditions of microbial-viral absence.

1. An ultrasound conducting gel comprising a gelling component, a disinfectant component, a humectant component, a base, disodium EDTA and water, wherein said disinfectant component is a non-alcoholic and non-volatile disinfectant, and wherein:
said gelling component is in an amount in the range of 0.5 to 4% w/w,
said disinfectant component is in an amount in the range of 0.01 to 10% w/w, and
said disodium EDTA is in an amount in the range of 0.01 to 0.3% w/w.

2. An ultrasound conducting gel comprising a gelling component, a disinfectant component, disodium EDTA, a humectant, a base, a dye and water, wherein said disinfectant component is a non-alcoholic and non-volatile disinfectant.

3. The ultrasound conducting gel according to claim 1, wherein:
said gelling component is in an amount in the range of 0.5 to 2% w/w,
said disinfectant component is in an amount in the range of 0.01 to 2% w/w, and
said disodium EDTA is in an amount in the range of 0.01 to 0.1% w/w.

4. The ultrasound conducting gel according to claim 1, wherein said gelling component is a polyacrylic acid polymer, said disinfectant component is benzalkonium chloride, said humectant component is glycerol and said base is sodium hydroxide.

5. The ultrasound conducting gel according to claim 1, further comprising a dye.

6. The ultrasound conducting gel according to claim 1, said gel comprising:
1.5% w/w Carbomer 940,
10% w/w glycerol,
0.1% w/w dye,
0.2% w/w benzalkonium chloride,
0.05% w/w disodium EDTA, and wherein said water is deionized water.

7. The ultrasound conducting gel according to claim 1, as an ultrasound transmitting agent.

8. The ultrasound conducting gel according to claim 7, wherein said ultrasounds are transmitted by ultrasound equipment.

9. The ultrasound conducting gel according to claim 6, as a transmitting agent for laser equipment.

10. The ultrasound conducting gel according to claim 1, as a transmitting agent for high intensity pulsed light (IPL) equipment.

11. Method of preventing transmission of bacteria, viruses and fungi with of the ultrasound conducting gel according to claim 1.

12. The method according to claim 11, wherein said virus is SARS-COV-2.
